(19) United States
(12) Patent Application Publication
(10) Pub. No.: US 2026/0352020 A1
(43) Pub. Date: Nov. 5, 2026

(54) A SYSTEM FOR POWERING A DEVICE AND METHOD OF THE SAME

(71) Applicant: University of Leeds, Leeds (GB)

(72) Inventors: Hemant PANDIT, Leeds (GB); Steven FREEAR, Leeds (GB); Bernard Hendrik VAN DUREN, Leeds (GB); Thomas Michael CARPENTER, Leeds (GB); David Matthew Joseph COWELL, Leeds (GB)

(73) Assignee: University of Leeds, Leeds (GB)

(21) Appl. No.: 18/997,687

(22) PCT Filed: Jul. 19, 2023
(86) PCT No.: PCT/GB2023/051900

§ 371 Date: Jan. 22, 2025

(30) Foreign Application Priority Data
Jul. 22, 2022 (GB) 2210780.9

(51) Int. Cl. H02J 50/10; H02J 50/15; H02J 50/20; H02J 50/40

(52) U.S. Cl. CPC H02J 50/10; H02J 50/15; H02J 50/20; H02J 50/40

(57) ABSTRACT

A system for powering a device, the system comprising: a wireless power transmitter configured to transmit a first power signal, and a receiver unit configured to: receive the first power signal, convert the first power signal to a second power signal for wirelessly powering a device, and transmit the second power signal to power the device through inductive coupling.

This invention relates to a system for powering a device, in particular, but not exclusively to charging an implantable device, and associated methods.

### BACKGROUND

When wirelessly charging a device, it is necessary to account for the different transmission environments (e.g. air or soft tissue) through which power is to be transmitted. When the device is in close proximity to an inductive charging module, there may be little or no air between the charging module and the charging circuit of the device. However, when wirelessly transmitting power over a larger distance, or through soft tissue in the case of implanted devices, the transmission environment necessitates optimisation of the antennae used to transmit wireless power.

For implanted devices, it is possible to rely on inductive charging to transmit power through the soft tissue (e.g. fat, muscle and bone) to power or recharge the implanted device when the device is placed in close proximity to the inductive charging module. However, in many situations an implanted device requires the patient to be static or for the wireless charger to remain at a fixed position relative to the device. This is undesirable if it may be necessary for the patient to remain tethered to a mains power supply or to carry a power source with them. As the patient moves within a space, the implanted device will not be in sufficiently close proximity to the wireless charger, which will render inductive charging impractical or impossible. More generally, it is difficult to wirelessly power a device which is moving within a space, due to the varying distance between the charging coil and the patient.

The present invention seeks to address at least some of these issues.

### BRIEF SUMMARY OF THE DISCLOSURE

A system for powering a device, the system comprising: a wireless power transmitter configured to transmit a first power signal, and a receiver unit configured to: receive the first power signal, convert the first power signal to a second power signal for wirelessly powering a device, and transmit the second power signal to power the device through inductive coupling.

The first power signal may be any of an acoustic signal, an ultrasonic signal or a microwave signal.

The wireless power transmitter may be configured to transmit the first power signal using a phased array.

The receiver unit may comprise a near-field transmitter configured to transmit the second power signal.

The second power signal may be a low voltage power signal, for example having a voltage of less than 10V and/or less than 10 mA.

The wireless power transmitter may be configured to receive a localisation signal indicative of a position of the receiver unit. The wireless power transmitter may be configured to direct the first power signal towards the receiver unit based on the localisation signal.

Viewed from a further independent aspect, there is also provided a system for powering a device, the system comprising: a wireless power transmitter configured to transmit a first power signal, and a receiver unit configured to receive the first power signal for powering a device operatively connected to the receiver unit, wherein the wireless power transmitter is configured to: receive a localisation signal indicative of a position of the receiver unit, and direct the first power signal towards the receiver unit based on the localisation signal.

The receiver unit may be configured to transmit the localisation signal. The localisation signal may be a Bluetooth Low Energy signal.

The wireless power transmitter may comprise an adaptive phased array transmitter. The wireless power transmitter may comprise a far-field transmitter configured to transmit the first power signal. The wireless power transmitter may be configured to transmit the first power signal in a plurality of directions.

The receiver may be embedded within a fabric layer. The charging system may comprise a garment. The garment may comprise the fabric layer.

The receiver unit may comprise an adhesive layer for mounting the receiver unit to an external surface. The adhesive layer may be configured to adhere the receiver unit to skin.

Viewed from a further independent aspect, there is also provided a receiver unit adapted for use in the charging system according to any preceding claim.

Viewed from a further independent aspect, there is also provided a method of wirelessly powering a device, the method comprising: wirelessly transmitting, from a wireless power transmitter, a first power signal to a receiver unit, converting, by the receiver unit, the first power signal to a second power signal for wirelessly powering a device, and powering the device using the second power signal by inductive coupling.

Viewed from a further independent aspect, there is also provided a method of wirelessly powering a device, the method comprising: receiving, by a wireless power transmitter, a localisation signal indicative of a position of a receiver unit, directing, by the wireless power transmitter, a first power signal towards the receiver unit based on the localisation signal.

The first power signal may be transmitted using a phase array.

The localisation signal may be a Bluetooth Low Energy signal.

The localisation signal may be transmitted from the receiver unit.

### BRIEF DESCRIPTION OF THE DRAWINGS

Embodiments of the invention are further described hereinafter with reference to the accompanying drawings, in which:

FIG. 1 is a schematic illustration of an exemplary system;

FIG. 2 is a schematic illustration of a second exemplary system;

FIG. 3 is a cross-sectional view of the system of FIG. 2;

FIG. 4 is a schematic illustration of a third exemplary system;

FIG. 5 is a cross-sectional view of the system of FIG. 4;

FIG. 6 is a schematic illustration of a fourth exemplary system;

FIG. 7 illustrates an exemplary method of powering a device;

FIG. 8 is a schematic illustration of a fifth exemplary system;

FIG. 9 illustrates a second exemplary method of powering a device.

### DETAILED DESCRIPTION

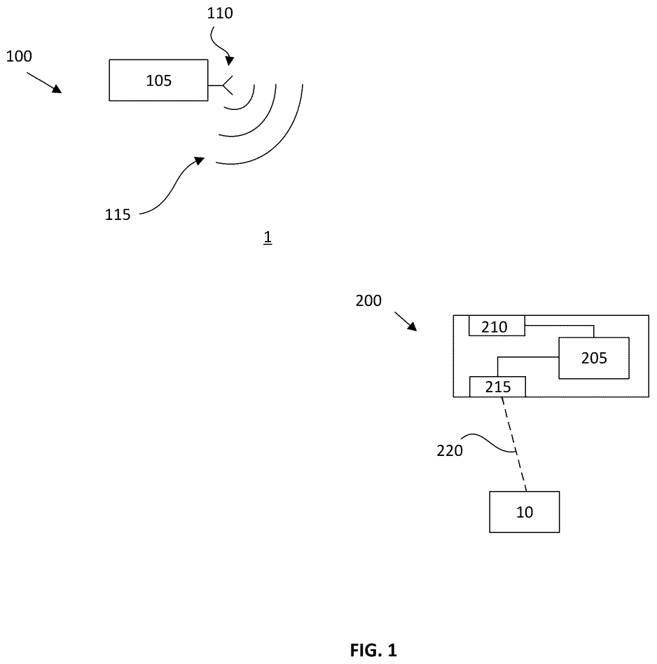

With reference to FIG. 1, there is provided an exemplary system 100 for powering a device 10. The illustrated system 100 includes a wireless power transmitter 105 having an antenna 110 for transmitting a first power signal 115. While a single antenna is shown, it would be apparent that a plurality of antennae 110 could be used, for example to provide a phased array power transmitter. The phased array power transmitter may be implemented as any of a dynamic phased array, a fixed phase array, an active phased array or a passive phased array as is known in the art.

The first power signal 115 is transmitted according to a first modality, for example ultrasound or microwave, through a first transmission environment 1 (e.g. air) and is received by a receiver unit 200. The wireless power transmitter 105 may radiate the first power signal 115 in all directions, or may generate a beam directed in a pre-determined direction. In some cases, the wireless power transmitter 105 can sweep the beam across an area. In some cases, the wireless power transmitter 105 is a far-field transmitter.

The receiver unit 200 includes a receiver antenna 210, a transmitter antenna 215 and a controller 205 operatively coupled to the receiver antenna 210 and the transmitter antenna 215. The transmitter antenna 215 can include a transmitter coil or similar to form an inductive coupling between the receiver unit 200 and the device 10 to send a second power signal 220 to wirelessly power the device 10. The receiver unit 200 converts the received power signal 220 into DC or AC electric current which can be used to power the transmitter coil 215. The second power signal 220 is transmitted according to a second transmission modality, different to the first modality. In some cases the receiver unit 200 includes a near-field transmitter to transmit the second power signal 220.

The present system uses two different transmission modalities to make use of the most appropriate transmission modality for each step. For example, transmission of the first power signal 115 through the air 1 can be considered a first step, and transmission of the second power signal 220 through soft tissue 5 can be considered a second step. While the present two-step approach is particularly suited for powering implanted devices as explained below, it would be apparent the present approach can be applied to non-implanted devices where power is to be transmitted across multiple different transmission environments, such as portable electronic devices. It would also be apparent that more than two different transmission modalities may be used to wirelessly transfer power to the device 10, particularly when different transmission modalities are more suited to transmit power through the different transmission environments.

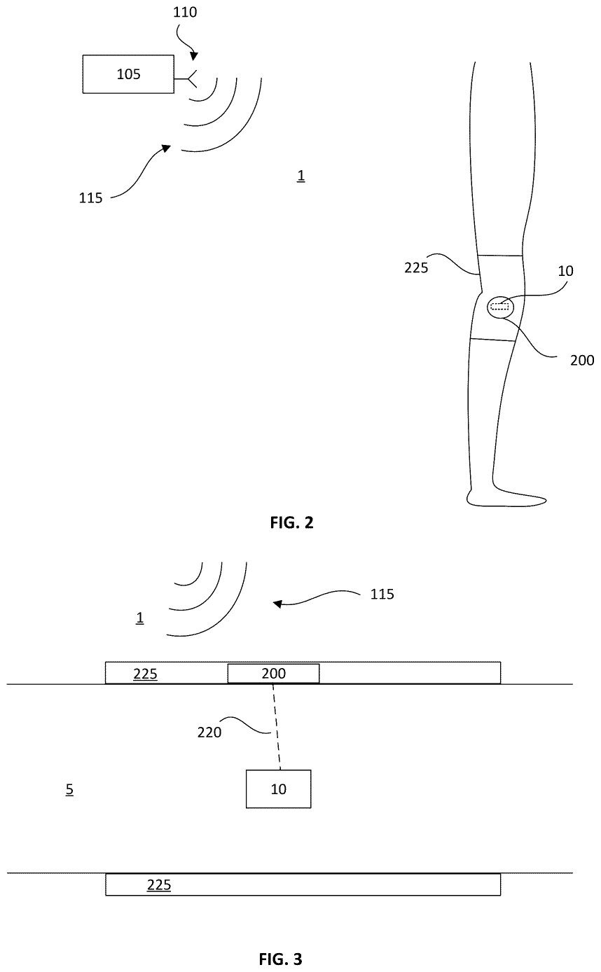

FIG. 2 illustrates the receiver unit 200 embedded in a fabric layer of a garment 225, for example as a sleeve or sock. In FIG. 2, the device is implanted in the patient and the garment 225 positions the receiver unit 200 over a lateral aspect of the knee in close proximity to the implanted device 10 and includes a rechargeable battery (not shown). Embedding the receiver unit 200 in a garment is advantageous as a patient can wear the garment 225 for prolonged periods of time, such as overnight, while the device 10 charges.

FIG. 3 shows the first power signal 115 passing through the first transmission environment (for example air 1) to reach the receiver unit 200, and the second power signal 220 passing through the soft tissue 5 of the patient's leg (a second transmission environment). In one example, a phased array microwave or ultrasonic signal is used to transmit the first power signal 115 to the receiver unit 200, and the receiver unit 200 converts this to an electromagnetic signal to inductively charge the device 10.

A phased power signal 115, for example a phased microwave or ultrasound signal, is particularly advantageous, as the first power signal 115 reaches the receiver unit 200 with sufficient power such that the receiver unit 200 can convert the first power signal 115 to inductively charge in the implanted device 10 via an electromagnetic coupling. This does away with the need to have a separate power supply for charging the implanted device 10 and provides a charging system with significantly more flexibility than existing systems for charging implanted devices. Preferably the receiver unit 200 has no separate power supply. While the power induced by the receiver unit 200 may be a low voltage signal, this is not an issue when the implanted device 10 will remain in the vicinity (e.g. 3-4 m) of the wireless power transmitter 105 for a prolonged period of time, such as overnight while the patient is asleep in an approximately fixed position relative to the wireless power transmitter 105. A low voltage signal will be typically a few Volts (e.g. less than 10V) and/or a few Milliamperes (e.g. less than 100 mA). The tolerances of the present system can accommodate movement of the patient within a bed within the room. While the implanted device 10 is shown implanted in the knee joint of the patient, it would be apparent this was merely exemplary, and that the present system could be used to power or charge a device 10 implanted at other locations in the body, or not implanted in the body, but placed on the body. As explained above, the device may be a portable electronic device, such as a mobile phone.

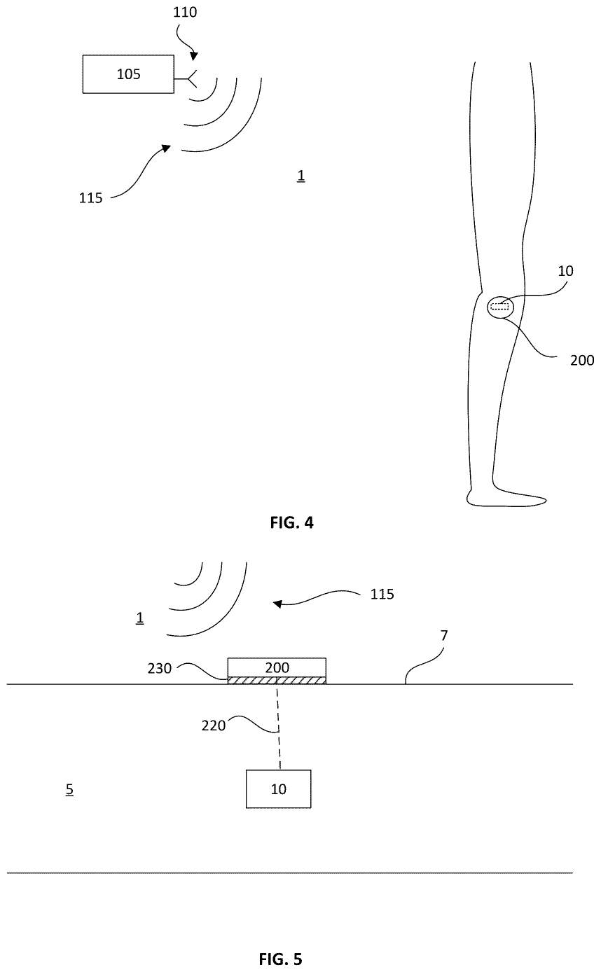

FIG. 4 is a schematic illustration of a third exemplary system where the receiver unit 200 has an adhesive layer 230 for sticking the receiver unit 200 to the skin surface 7 of the patient (see also FIG. 5). A receiver unit 200 having an adhesive layer 230 can be easily provided as a patch which can be easily applied to the skin surface 7 in the vicinity of the implanted device 10. This ensures the receiver unit 200 remains in a relatively fixed position relative to the implanted device 10 as the patient moves relative to the wireless power transmitter 105.

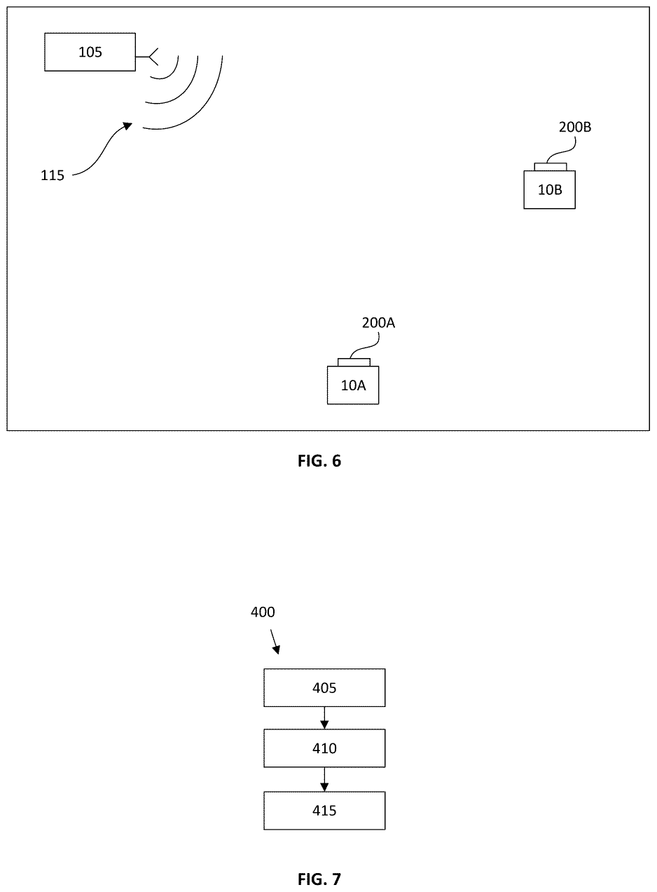

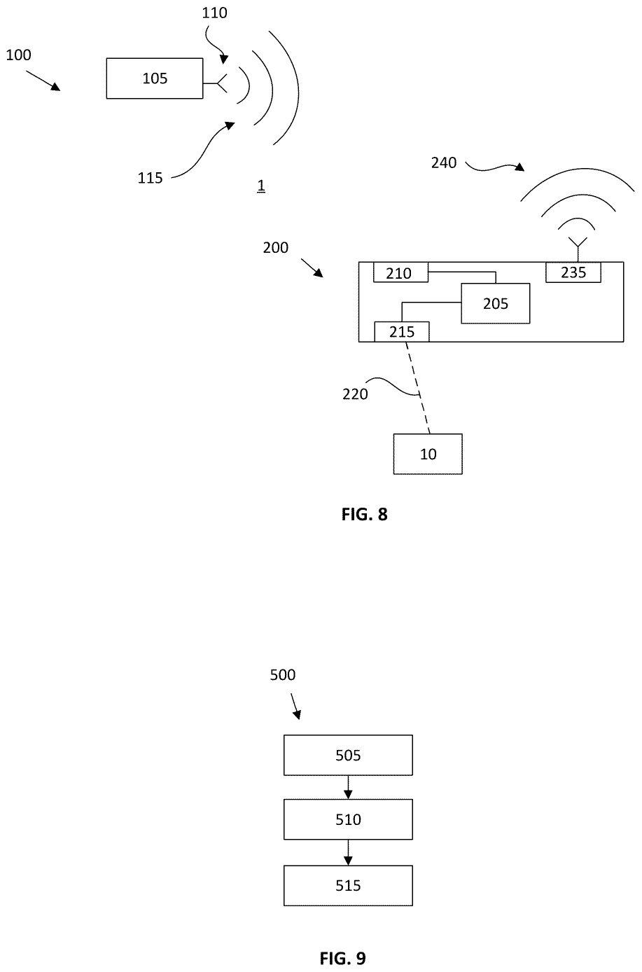

FIG. 6 is a schematic illustration of a fourth exemplary system where multiple devices 10A, 10B are moving within a room. Each of the devices 10A, 10B has a respective receiver unit 200A, 200B secured thereto, for example using a layer of material (such as in a garment 225) or adhesive layer 230 to fix the receiver unit 200 to the device 10 as described above. As the devices 10A, 10B remain in the room, they remain relatively close, e.g. within 4 m, to the wireless power transmitter 105. In this case, the wireless power transmitter 105 is able to power or charge both devices 10A, 10B simultaneously.

FIG. 7 illustrates an exemplary method 400 of powering a device 10. The method 400 includes wirelessly transmitting 405 a first power signal 115 to the receiver unit 200, converting 410 the first power signal 115 to a second power signal 220 for wirelessly powering the device 10, and powering 415 the device 10 using the second power signal 220 by inductive coupling. By way of example, the device 10 may be a sensor-based implanted device. In some cases, the device 10 includes a rechargeable battery, for example having a capacity of 25 mAh, and a charging circuit for charging the rechargeable battery.

In some cases the receiver unit 200 includes multiple charging coils (not shown) for powering the device 10. While a charging system 100 is described herein, it would be apparent in some cases the same system 100 may be for powering devices without a rechargeable battery.

In some cases, the wireless power transmitter 105 can be mounted to a wall of a room, or be placed on or secured to a structure within the room. By way of example, the wireless power transmitter 105 can be secured to a bed frame of a patient wearing a garment containing the receiver unit 200 as described above. In this case, the first power signal 115 is transmitted through any bedding and the mattress on the bed frame to the garment 225. This distance is typically too far to transmit power via inductive coupling, but is possible using the far-field techniques described herein. The first power signal 115 can be converted to power the transmitter antenna 215 to transmit the second power signal 220 to the implanted device 10 via the inductive coupling which is closer to the receiver unit 200.

The receiver unit 200 can also transmit a localisation signal 240 from a Bluetooth module 235, preferably a Bluetooth Low Energy unit. The localisation signal 240 can be detected by the wireless power transmitter 105, and the first power signal 115 can be directed towards the source of the localisation signal 240 (i.e. beamforming). This localisation guided beamforming advantageously increases the power transferred to the receiver unit 200 compared to a radiated first power signal 115. While received signal strength indicator (RSSI) is a form of localisation signal, it would be apparent this was not essential, and other localisation signals or localisation parameters can be used additional or alternatively to this. Similarly, while the receiver unit 200 is described as providing the localisation signal 240, this is not essential, as the Bluetooth module 235 may be part of the device 10 being powered, or may be a standalone device separate from the receiver unit 200 or the device 10 being powered. It would also be apparent that multiple devices may provide localisation signal 240 (i.e. any combination of the device 10, the receiver unit 200 and the standalone device).

In some cases, the receiver unit 200 does not convert the first power signal 115 to a second power signal 220 of a different modality to the first power signal 115. That is to say, the second power signal 220 may be the same modality as the first power signal 115. This may be useful in cases where the receiver unit 200 is used to direct the first power signal 115 towards itself such that the charging efficiency of the device 10 can be increased. When multiple devices 10A, 10B are present in a space, each device 10A, 10B can be localised in the manner described herein to provide multiple beams for powering each device 10A, 10B.

FIG. 9 is a schematic representation of an alternative method 500 of powering a device 10. The method 500 includes the step of transmitting 505 the first power signal 115 in a first direction, receiving 510 a localisation signal 240 indicative of a position of the device 10 (e.g. transmitted from the device 10 itself, a standalone device (not shown), or the receiver module 200), directing 515 the first power signal 115 based on the localisation signal 240 (e.g. towards the source of the localisation signal 240 which may be any of the device 10, a standalone device, or the receiver module 200). The directed first power signal 115 may be in a second direction different to the first direction. Where multiple devices 10A, 10B are present, it would be apparent that the signal for powering a respective device (e.g. device 10A) can be directed independently of the signals for powering the remaining devices (e.g. device 10B). Step 505 is not essential, as the wireless power transmitter 105 may only start transmitting the first power signal after receipt of the localisation signal 240.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

What is claimed is:

1. A system for powering a device, the system comprising:
a wireless power transmitter configured to transmit a first power signal, and a receiver unit configured to:
receive the first power signal,
convert the first power signal to a second power signal for wirelessly powering a device, and
transmit the second power signal to power the device through inductive coupling.

2. A charging system according to claim 1, wherein the first power signal is any of an acoustic signal, an ultrasonic signal or a microwave signal.

3. A charging system according to claim 1, wherein the wireless power transmitter is configured to transmit the first power signal using a phased array.

4. A charging system according to claim 1, wherein the receiver unit comprises a near-field transmitter configured to transmit the second power signal.

5. A charging system according to claim 1, wherein the second power signal is a low voltage power signal.

6. A charging system according to claim 1, wherein the wireless power transmitter is configured to receive a localisation signal indicative of a position of the receiver unit, and wherein the wireless power transmitter is configured to direct the first power signal towards the receiver unit based on the localisation signal.

7. (canceled)

8. A charging system according to claim 6, wherein the receiver unit is configured to transmit the localisation signal.

9. A charging system according to claim 6, wherein the localisation signal is a Bluetooth Low Energy signal.

10. A charging system according to claim 1, wherein the wireless power transmitter comprises an adaptive phased array transmitter.

11. A charging system according to claim 1, wherein the wireless power transmitter comprises a far-field transmitter configured to transmit the first power signal.

12. A charging system according to claim 1, wherein the wireless power transmitter is configured to transmit the first power signal in a plurality of directions.

13. A charging system according to claim 1, wherein the receiver is embedded within a fabric layer.

14. A charging system according to claim 1 comprising a garment, and wherein the garment comprises the fabric layer.

15. A charging system according to claim 1, wherein the receiver unit comprises an adhesive layer for mounting the receiver unit to an external surface.

16. A charging system according to claim 15, wherein the adhesive layer is configured to adhere the receiver unit to skin.

17. A receiver unit adapted for use in the charging system according to claim 1.

18. A method of wirelessly powering a device, the method comprising:
wirelessly transmitting, from a wireless power transmitter, a first power signal to a receiver unit,
converting, by the receiver unit, the first power signal to a second power signal for wirelessly powering a device, and
powering the device using the second power signal by inductive coupling.

19-21. (canceled)

22. A method according to claim 18, wherein the first power signal is transmitted using a phase array.
